(19) United States
(12) Patent Application Publication
(10) Pub. No.: US 2013/0030406 A1
(43) Pub. Date: Jan. 31, 2013

(54) Textured Dilatation Balloon and Methods

(71) Applicants: Susheel Deshmukh, Santa Rosa, CA (US); Kishore Udipi, Santa Rosa, CA (US); Josiah Wilcox, Santa Rosa, CA (US)

(72) Inventors: Susheel Deshmukh, Santa Rosa, CA (US); Kishore Udipi, Santa Rosa, CA (US); Josiah Wilcox, Santa Rosa, CA (US)

(73) Assignee: Medtronic Vascular, Inc., Santa Rosa, CA (US)

(21) Appl. No.: 13/190,667

(22) Filed: Jul. 26, 2011

(51) Int. Cl. A61M 29/02; B05D 3/10; A61L 31/16

(52) U.S. Cl. 604/500; 604/103.02; 427/2.14

(57) ABSTRACT

The present disclosure provides a textured dilatation balloon that includes a balloon body having a proximal end, a distal end, and at least one indentation in the balloon body in an un-inflated state, wherein the balloon body comprises a continuous polymer tube with an external surface having at least one therapeutic agent disposed within the at least one indentation.

### BACKGROUND OF THE DISCLOSURE

Surgical procedures employing balloons and medical devices incorporating those balloons (i.e., balloon catheters) are becoming more common and routine. These procedures, such as angioplasty procedures, are conducted when it becomes necessary to expand or open narrow or obstructed openings in blood vessels and other passageways in the body to increase the flow through the obstructed areas. For example, in an angioplasty procedure, a dilatation balloon catheter is used to enlarge or open an occluded blood vessel which is partially restricted or obstructed due to the existence of a hardened stenosis or buildup within the vessel. This procedure requires that a balloon catheter be inserted into the patient's body and positioned within the vessel so that the balloon, when inflated, will dilate the site of the obstruction or stenosis so that the obstruction or stenosis is minimized, thereby resulting in increased blood flow through the vessel.

Many times, once the balloon has been arranged at the vessel narrowing, it is repeatedly inflated and deflated. The inflation, with successive deflation, of the balloon within the vessel (e.g., artery) reduces the extent of the vessel luminal narrowing, and restores a suitable blood flow in the cardiac area suffering from the stenosis. In some cases, the balloon serves to deliver a stent.

In both cases, after a few months, some patients develop a new narrowing of the vessel wall at the intervention point. Such narrowing, known under the name of restenosis, is not due to the formation of new atherosclerotic plaques, but to a cell hyper-proliferation process, particularly of the vascular smooth muscle cells, probably due to the dilating action operated by the foreign body, stent or balloon.

It has been observed that restenosis can be treated by coating a stent or a balloon with a drug, e.g., having anti-proliferative action. Such a stent is often referred to as a “drug eluting stent” (DES) and such a balloon is referred to as a “drug eluting balloon” (DEB). For various reasons, the use of a drug eluting balloon is preferred over the use of a drug eluting stent. Control of the delivery, whether it be immediate or over time, from a balloon, for example, is a challenge, however.

### SUMMARY

The present disclosure provides textured dilatation balloons, methods of making, and methods of using. The textured balloons are preferably non-compliant or semi-compliant.

In one embodiment, a textured dilatation non-compliant or semi-compliant balloon includes a balloon body having a proximal end, a distal end, and at least one indentation in the balloon body in an un-inflated state, wherein the balloon body includes a continuous polymer tube with an external surface having at least one therapeutic agent disposed in the at least one indentation prior to use.

In certain embodiments, a textured dilatation balloon includes a plurality of indentations (i.e., depressions). Such one or more indentations can be in a variety of shapes, sizes, and/or volumes. For example, they can be in the form of circular indentations, e.g., dimples, grooves, inverted pyramids, inverted square pyramids, and the like, or combinations thereof in any one balloon. Alternatively, a balloon body can include one continuous indentation, e.g., a continuous channel.

The control of the shape(s), size(s), and/or volume(s) of the one or more indentations, as well as the number and location of the indentations, assist in the control of the volume of therapeutic agent disposed therein for delivery to a target site (e.g., a vessel wall). Significantly, the balloons of the present disclosure can provide less loss of therapeutic agent during insertion of the balloon to the target site, and less non-specific release at the target site.

In certain embodiments, the external surface of the continuous tube of the balloon body further includes at least one organic polymer disposed thereon. In certain embodiments, the at least one therapeutic agent is located within the at least one indentation and the at least one organic polymer is disposed over the at least one therapeutic agent (e.g., as a cap coat). In certain embodiments, the at least one therapeutic agent is mixed with the at least one organic polymer to form a mixture that is disposed within the at least one indentation. In certain embodiments, the at least one therapeutic agent is mixed with at least one excipient to form a mixture that is disposed within the at least one indentation.

In certain preferred embodiments, the at least one therapeutic agent, optionally mixed with an organic polymer and/or an excipient, is disposed only in the at least one indentation

The present disclosure also provides methods using the dilatation balloons of the present disclosure. In one embodiment, a method of delivering at least one therapeutic agent to a target site in a patient is provided. The method includes: providing a textured non-compliant or semi-compliant dilatation balloon as described herein, such as one comprising: a balloon body having a proximal end, a distal end, and at least one indentation in the balloon body in an un-inflated state, wherein the balloon body comprises a continuous polymer tube with an external surface having at least one therapeutic agent disposed within the at least one indentation; inserting a balloon catheter comprising the textured dilatation balloon into the target site of the patient; and inflating the textured balloon at the target site under conditions effective to flatten the at least one indentation and deliver at least a portion of the therapeutic agent(s) to the target site.

Preferably, a “therapeutically effective amount” is delivered to the target site (e.g., a vessel wall). By this it is meant an amount capable of inducing a therapeutic or preventive effect against the restenosis of the treated vascular tissue in the patient.

In another embodiment, the present disclosure provides a method of making a dilatation balloon. The method typically includes a blow molding process. In one embodiment, a method includes: providing a tubular parison comprising a polymeric material; providing a mold having one or more protrusions on its inner surface corresponding to the desired texture of the balloon surface; expanding the tubular parison to form an expanded parison in the mold and form a balloon body comprising one or more indentations; and applying one or more therapeutic agents into the one or more indentations. Preferably, the balloon is a non-compliant or semi-compliant balloon. In certain preferred embodiments, expanding the tubular parison to form an expanded parison comprises axially stretching and radially expanding the tubular parison at a temperature above the Tg of the polymeric material and at an elevated inflation pressure.

Herein, the terms “distal” and “proximal” are used with respect to a position or direction relative to the treating clinician. “Distal” and “distally” are a position distant from or in a direction away from the clinician. “Proximal” or “proximally” are a position near or in a direction toward the clinician.

Herein, an “Indentation in the balloon body in an un-inflated state” means a predesigned surface cavity in the balloon surface of a specific size. An indentation does not result from two separate balloons proximal and distal to the target site. Such balloons with indentations are not weeping balloons. Such indentations are not folds as a result of balloon folding. The one or more indentations are actually in the material that forms the balloon. They are not pores (e.g., expandable pores). They are not in a porous retaining material, such as a porous matrix, sheet, or bundle of fibers, that forms an outer layer or sleeve on a balloon, nor are they pores directly formed in the balloon surface, as described in U.S. Pat. Pub. No. 2008/0140002.

Herein, “prior to use” refers to the state of the balloon prior to the balloon being inserted into the target site of the patient.

“Fully inflated” means the balloon is inflated to a state where the indentations are flattened out and the material contained therein (one or more therapeutic agents optionally mixed with an organic polymer and/or an excipient) is exposed to the target site (e.g., vascular tissue) and transferred thereto.

The terms “comprises” and variations thereof do not have a limiting meaning where these terms appear in the description and claims.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

As used herein, “a,” “an,” “the,” “at least one,” and “one or more” are used interchangeably.

As used herein, the term “or” is generally employed in its sense including “and/or” unless the content clearly dictates otherwise. The term “and/or” means one or all of the listed elements or a combination of any of the listed elements.

Also herein, all numbers are assumed to be modified by the term “about” and preferably by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used.

The recitations of numerical ranges by endpoints include all numbers subsumed within that range as well as the endpoints (e.g., 1 to 5 includes 1, 1.5, 2, 2.75, 3, 3.80, 4, 5, etc.).

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

FIG. 1 shows a detailed section of a balloon segment of the present disclosure showing indentations.

FIG. 2 shows the balloon catheter with balloon in an un-inflated state showing indentations.

FIG. 3 shows the distal section (100) of the balloon catheter with the balloon in an un-inflated state showing one indentation.

FIG. 4 shows the balloon catheter in an un-inflated state.

FIG. 5 shows the distal section of the balloon catheter with the balloon in an un-inflated state.

### DETAILED DESCRIPTION OF ILLUSTRATIVE EMBODIMENTS

The present disclosure provides textured dilatation balloons, methods of making, and methods of using. In one embodiment, the textured dilatation balloon includes a balloon body having a proximal end, a distal end, and at least one indentation in the balloon body in an un-inflated state, wherein the balloon body includes a continuous polymer tube with an external surface having at least one therapeutic agent disposed in the at least one indentation prior to use.

In certain embodiments, the textured dilatation balloon includes a plurality of indentations (i.e., depressions, cavities, or craters), such as dimples, when in the un-inflated state. Alternatively, a balloon body can include one continuous indentation, e.g., a continuous channel or trough, when in the un-inflated state. The control of the shapes, sizes, and/or volumes of the one or more indentations, as well as the number and location of the indentations, assist in the control of the volume of therapeutic agent disposed therein for delivery to a vessel wall.

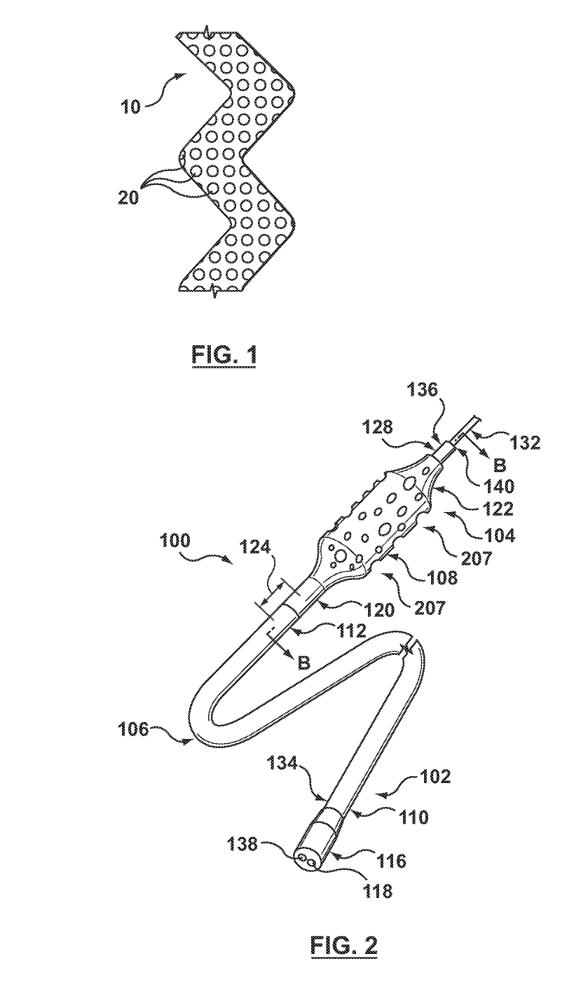

Referring to FIG. 1, an exemplary embodiment is shown in which an un-inflated balloon segment (10) has circular indentations (20) (e.g., dimples) in the balloon body surface. During transport, when the balloon is in an un-inflated or deflated state, the openings in the balloon surface that provide access into the indentations can be closed, thereby creating reservoirs for holding the one or more therapeutic agents on the surface of the balloon. Upon inflation of the balloon, the openings in the balloon surface that provide access into the indentations are opened. That is, the force with which the inflated (i.e., expanded) condition of the balloon exerts radially will open the indentations and flatten them out, releasing the therapeutic agent(s) to the target site. The indentations are essentially eliminated upon inflation. This provides for better control of the delivery of a specific amount of one or more therapeutic agents to a target site.

The one or more indentations can be in a variety of shapes, sizes, and/or volumes. For example, they can be in the form of circular indentations, e.g., dimples, grooves, inverted pyramids, inverted square pyramids, inverted cones, wells, and the like, or combinations thereof in any one balloon. A plurality of indentations can be evenly or unevenly, symmetrically or unsymmetrically, spaced on a balloon surface.

Alternatively, a balloon body can include one continuous indentation, e.g., a continuous channel or trough. The control of the shape(s), size(s), and/or volume(s) of the one or more indentations, as well as the number and location of the indentations, assist in the control of the volume of therapeutic agent(s) disposed therein for delivery to a target site, e.g., vessel wall.

Upon inflation of the balloon and contact with tissue at the target site, e.g., a vessel wall, at least a portion of the one or more therapeutic agents (preferably, a therapeutically effective amount) is transferred to the tissue. The indentation(s) can include different therapeutic agents in different regions of the balloon surface. Alternatively or additionally, the indentation(s) can include the same therapeutic agent at different concentrations. Alternatively or additionally, the indentation(s) can include different therapeutic agents in a layered configuration. In this way, for example, a first agent may be delivered when the balloon reaches its first diameter, a second agent may be delivered upon further inflation, and a third agent may be delivered upon yet even further inflation.

In certain embodiments, the total area of the two-dimensional (surface) opening of the at least one indentation is at least 20%, at least 30%, at least 40%, at least 50%, or at least 60%, of the surface area of the external surface of the continuous tube of the balloon body in an un-inflated state. By this it is meant that the “total area” is the summation of the areas of the two-dimensional (surface) openings of all the indentations. In certain embodiments, the total area of the two-dimensional (surface) opening of the at least one indentation is no greater than 90%, no greater than 80%, no greater than 70%, no greater than 60%, no greater than 50%, of the surface area of the external surface of the continuous tube of the balloon body in an un-inflated state.

Balloons of the present disclosure have a balloon body that includes a continuous polymer tube. In certain embodiments, the balloon body includes a plurality of indentations. The spacing and arrangement of the indentations can be of any desired spacing or arrangement. For example, the length of the balloon body (in an un-inflated state) between the indentations (i.e., the continuous polymer tube) can be at least 6 mm in length. In certain embodiments, the length of the balloon body between the indentations can be no more than 30 mm in length. The indentations can be symmetrically or unsymmetrically arranged, typically symmetrically arranged, on the external surface of the continuous tube of the balloon body. If desired, the indentations can be distributed evenly over the entire external surface of the continuous tube of the balloon body.

The dimensions of the balloons of the present disclosure can be those typically used for coronary, peripheral, and valvuloplasty balloons.

The diameter of the balloon body at each indentation (in an un-inflated state) may be the same or different. For example, the diameter of the balloon body at the indentations is at least 0.4 mm in diameter smaller, and more preferably no more than 0.5 mm in diameter smaller, than the balloon body diameter between the indentations.

Preferably, a balloon of the present disclosure has a wall thickness that ranges from 0.0003 inch to 0.003 inch. Balloons of the present disclosure have a balloon body between the indentations that includes a continuous polymer tube with a wall thickness that is typically the same as that of the indentations in a deflated state (i.e., un-inflated state). In certain embodiments, the wall thickness of the balloon body (in a deflated state) in the regions that are not indented (e.g., between the indentations) is at least 0.012 mm. In certain embodiments, the wall thickness of the balloon body (in a deflated state) in the regions that are not indented (e.g., between the indentations) is no more than 0.025 mm. When inflated to nominal pressure, the indentations disappear.

Balloons of the present disclosure are preferably non-compliant or semi-compliant. This classification is based upon the operating characteristics of the individual balloon, which in turn depend upon the process used in forming the balloon, as well as the material used in the balloon forming process. All types of balloons provide advantageous qualities. A balloon which is classified as “non-compliant” is characterized by the balloon's inability to grow or expand appreciably beyond its rated or nominal diameter. Non-compliant balloons are referred to as having minimal distensibility. In balloons currently known in the art (e.g., polyethylene terephthalate), this minimal distensibility results from the strength and rigidity of the molecular chains which make up the base polymer, as well as the orientation and structure of those chains resulting from the balloon formation process.

A balloon which is referred to as being “compliant” is characterized by the balloon's ability to grow or expand beyond its nominal or rated diameter. In balloons currently known in the art (e.g., polyethylene, polyvinylchloride), the balloon's compliant nature or distensibility results from the chemical structure of the polymeric material used in the formation of the balloon, as well as the balloon forming process. Compliant balloons upon subsequent inflations, will achieve diameters which are greater than the diameters which were originally obtained at any given pressure during the course of the balloon's initial inflation.

A balloon which is referred to as being “semi-compliant” is characterized by low compliance with moderate stretching upon the application of tensile force. Typically, a semi-compliant balloon has a compliance of less than 0.045 millimeters/atmosphere (mm/atm), whereas a compliant balloon has a compliance of greater than 0.045 mm/atm, and a non-compliant balloon has a compliance of not greater than 0.025 mm/atm. Examples of such semi-compliant balloon materials include Nylon 12 and Pebax 7033.

Preferred balloons of the present disclosure have high elasticity and high elastic recovery. Preferably, the balloon returns to approximately the same profile it had before the initial inflation.

The term “elastic,” as it is used in connection with this disclosure, refers only to the ability of a material to follow the same stress-strain curve upon the multiple applications of stress. Elasticity, however, is not necessarily a function of how distensible a material is. It is possible to have an elastic, non-distensible material or a nonelastic, distensible material.

Before initial inflation and when deflated, balloons of the present disclosure preferably have a much lower profile than wrapped conventional balloons, and can have essentially the same dimensions as the tubular pre-form. When inflated, balloons of the present disclosure transition from a low profile tube to a balloon having indentations at the proximal and distal ends. They preferably revert to the initial tubular form when deflated, even after multiple inflations and after multiple lesions have been dilated. Balloons of the present disclosure can have elasticity at nominal strains of at least 30%. Alternatively, balloons of the present disclosure can have elastic recovery from nominal strains equal to, or greater than, 30%, 40%, 50%, 60%, 70%, 80%, 90%, or 100%, where nominal strain is [(balloon o.d. at nominal pressure-preform o.d.)/preform o.d.]×100, where “o.d.” is the outer diameter. Preferred balloons of the present disclosure may, therefore, be used to dilate multiple lesions without compromising primary performance.

Materials used in balloons of the present disclosure are primarily thermoplastics or thermoplastic elastomers. They may be block co-polymers, graft co-polymers, a blend of elastomers and thermoplastics, and the like. Such polymers may be crosslinked or not, but preferably are not crosslinked. Various combinations of polymers may be used in making balloons of the present disclosure. For example, in balloons described herein, the continuous polymer tube of the balloon body can include one or more materials selected from the group consisting of a polyester homopolymer, a polyester copolymer, a polyamide homopolymer, a polyamide copolymer, a polyethylene homopolymer, a polyethylene copolymer, a polyurethane, a polyurethane copolymer, and combinations thereof. Typically, and preferably, such polymers are block copolymers. Examples of mixtures of polymers include mixtures of nylon and polyamide block copolymers and polyethylene terephthalate and polyester block copolymers.

For example, the polymers may include polyethylene terephthalate polymers and polybutylene terphthalate polymers. Other useful materials include polyesterether and polyetherester amide copolymers such as those described in U.S. Pat. No. 5,290,306 (Trotta et al.), polyether-polyamide copolymers such as those described in U.S. Pat. No. 6,171,278 (Wang et al.), polyurethane block copolymers such as those described in U.S. Pat. Nos. 6,210,364 B1, 6,283,939 B1, and 5,500,180 (all to Anderson et al.). Suitable polymers also include materials such as the multiblock copolymers of the zero-fold balloon described in U.S. Pat. Pub. No. 2005/0118370.

Particularly preferred non-compliant and semi-compliant balloons include (wherein the continuous polymer tube of the balloon body comprises) a polyethylene terephthalate, a polybutylene terephthalate, a polyamide, a polyether block amide, a polyblend comprising a polyamide, a polyblend comprising a polyethylene terephthalate, a polyblend comprising a polybutylene terephthalate, a multi-layer construction comprising a polyamide layer, a multi-layer construction comprising a polyethylene terephthalate layer, or a multi-layer construction comprising a polybutylene terephthalate layer.

In the present disclosure, at least one therapeutic agent is disposed in at least one indentation in the balloon body in an un-inflated state. In certain embodiments, the at least one therapeutic agent is disposed only in the at least one indentation, as opposed to on the external surface adjacent the at least one indentation (e.g., as opposed to on the external surface between indentations).

A suitable therapeutic agent for use herein is one that is capable of producing a beneficial effect against one or more conditions including inflammation, coronary restenosis, cardiovascular restenosis, angiographic restenosis, arteriosclerosis, hyperplasia, and other diseases and conditions. For example, the therapeutic agent can be selected to inhibit or prevent vascular restenosis, a condition corresponding to a narrowing or constriction of the diameter of the bodily lumen.

A suitable therapeutic agent for use herein is one that is capable of producing a beneficial effect against one or more conditions including inflammation, vascular stenosis, angioplasty restenosis, stent restenosis, arteriosclerosis, atherosclerosis, arteritis, vascular lesion development associated with any type of vascular injury or in the prevention, treatment of vulnerable plaques, and other diseases and conditions. For example, the therapeutic agent can be selected to inhibit or prevent vascular restenosis, a condition corresponding to a narrowing or constriction of the diameter of the vascular lumen where balloon angioplasty has been performed or a stent placed. Additionally, such a therapeutic agent could be used to treat vascular stenosis caused by atherosclerosis or other vascular diseases in association with balloon dilatation of the lesion site.

Examples of therapeutic agents include, but are not limited to, an antiangiogenesis agent, an antirestenotic agent, an anticoagulant, an antiendothelin agent, an antimitogenic factor, an antioxidant, an antiplatelet agent, an antibiotic, an anti-inflammatory agent, an antiproliferative agent, an mTor inhibitor, an antineoplastic agent, an antisense oligonucleotide, an antithrombogenic agent, a gene therapy agent, a calcium channel blocker, a clot dissolving enzyme, a growth factor, a growth factor inhibitor, a nitric oxide releasing agent, a vasodilator, a virus-mediated gene transfer agent, a compound that affects microtubule development, a cell cycle inhibitor, an inhibitors of smooth muscle proliferation, an endothelial cell growth factor, a reverse cholesterol transport agonist, a reverse cholesterol transport antagonist, and combinations of the above.

Specific examples of therapeutic agents include abciximab, angiopeptin, colchicine, eptifibatide, heparin, hirudin, lovastatin, methotrexate, streptokinase, paclitaxel, rapamycin, everolimus, deforolimus, zotarolimus, ticlopidine, tissue plasminogen activator, trapidil, urokinase, MCP-1 antagonists, TNF alpha inhibitors, dexamethasone, flucinolone, vinblastine, and growth factors and growth factor inhibitors for VEGF, TGF-beta, IGF, PDGF, FGF, and combinations thereof.

The balloon construction, including the selection of the one or more therapeutic agents, is selected such that the one or more therapeutic agents is released from the balloon to the vessel wall in the very short contact time available during an angioplasty procedure, for example, from a few seconds to one minute. Once the one or more therapeutic agents have been released, at least a portion is absorbed by the cell wall, before the blood flow washes it off. Ideally, it is therefore desirable that absorption of the one or more therapeutic agents occurs concomitantly to the release thereof from the balloon. However, it is just as well necessary that the one or more therapeutic agents are retained by the balloon surface in a manner sufficient to resist all the handling operations to which they are subjected, both during the production step and during the preparation and carrying out of the angioplasty procedure, in any case, before the balloon reaches the treatment site.

The one or more therapeutic agents can be mixed with low (less than 10,000 g/mole) to medium (10,000 to 25,000 g/mole) weight average molecular weight excipients that include a fatty acid ester of polyethylene glycol, a polyethylene glycol-polyester block copolymer, a fatty acid mono- or di-ester of glycerol, a fatty acid mono-, di-, or poly-ester of trimethylol ethane or trimethylol propane or pentaerythritol, a sugar, a water-soluble polyol. Also included within the term “excipient” are cyclodextrins, clathrates (cage compounds), sometimes referred to as spacer molecules like urea, crown ethers, deoxycholic acid, and cryptands. Various combinations of these can be used if desired. In certain embodiments, the at least one therapeutic agent is mixed with at least one excipient to form a mixture that is disposed within the at least one indentation.

To further assist in the control of the retention and release of the one or more therapeutic agents, in certain embodiments, the external surface of the continuous tube of the balloon body can further include at least one organic polymer disposed thereon. In certain embodiments, the at least one therapeutic agent is located within the at least one indentation and the at least one organic polymer is disposed over the at least one therapeutic agent (e.g., as a cap coat). In certain embodiments, the at least one therapeutic agent is mixed with at least one organic polymer to form a mixture that is disposed within the at least one indentation.

The one or more therapeutic agents can be mixed with, incorporated within, encased or enclosed within, a therapeutic agent carrier, for example, that can be made of one or more synthetic organic polymers, natural organic polymers, or combinations (e.g., copolymers, mixtures, blends, layers, complexes, etc.) of these. The polymers may be biodegradable or non-biodegradable. They may be hydrophilic or hydrophobic. In certain embodiments, the polymers are preferably hydrophilic. In certain embodiments, the polymers are preferably biodegradable.

Protection of the therapeutic agents can also occur through the use of an inert molecule (e.g., in a cap- or over-coating over the one or more therapeutic agents) that prevents access to the one or more therapeutic agents. For example, a coating of the one or more therapeutic agents can be over-coated readily with an enzyme, which causes either release of the therapeutic agents or activates the therapeutic agents.

In some embodiments, a therapeutic agent/carrier formulation (e.g., a therapeutic agent with an organic polymer cap-coat overcoating it, or a therapeutic agent/organic polymer mixture therewith) is preferably adapted to exhibit a combination of physical characteristics such as biocompatibility, and, in some embodiments, biodegradability and bio-absorbability, while providing a delivery vehicle for release of the one or more therapeutic agents. For example, the formulation is preferably biocompatible such that it results in no induction of inflammation or irritation when implanted, degraded or absorbed.

Biodegradable materials include synthetic polymers such as polyesters, polyethers, polyanhydrides, poly(ortho)esters, polyketals, polyamino acids, poly(butyric acid), tyrosine-based polycarbonates, poly(ester amide)s such as based on 1,4-butanediol, adipic acid, and 1,6-aminohexanoic acid, poly(ester urethane)s, poly(ester anhydride)s, poly(ester carbonate)s such as tyrosine-poly(alkylene oxide)-derived poly(ether carbonate)s, polyphosphazenes, polyurethanes such as those based on polyamino acids, polyarylates such as tyrosine-derived polyarylates, poly(ether ester)s such as, poly(epsilon-caprolactone)-block-poly(ethylene glycol)) block copolymers, and poly(ethylene oxide)-block-poly(hydroxy butyrate) block copolymers.

Biodegradable polyesters, include, for example, poly(glycolic acid) (PGA), poly(lactic acid) (PLA), poly(glycolic-co-lactic acid) (PGLA), poly(1,4dioxanone), poly(caprolactone) (PCL), poly(3-hydroxybutyrate) (PHB), poly(3-hydroxyvalerate) (PHV), poly(hydroxy butyrate-co-hydroxy valerate), poly(lactide-co-caprolactone) (PLCL), poly(valerolactone) (PVL), poly(tartronic acid), poly(beta-malonic acid), poly(propylene fumarate) (PPF) (preferably photo cross-linkable), poly(ethylene glycol)/poly(lactic acid) (PELA) block copolymer, poly(L-lactic acid-epsilon-caprolactone) copolymer, poly(trimethylene carbonate), poly(butylene succinate), and poly(butylene adipate).

Biodegradable polyanhydrides include, for example, poly[1,6-bis(carboxyphenoxy)hexane], poly(fumaric-co-sebacic)acid or P(FA:SA), and such polyanhydrides used in the form of copolymers with polyimides or poly(anhydrides-coimides) such as poly-[trimellitylimidoglycine-co-bis(carboxyphenoxy)hexane], poly[pyromellitylimidoalanine-co-1,6-bis(carboph-enoxy)-hexane], poly[sebacic acid-co-1,6-bis(p-carboxyphenoxy)hexane] or P(SA:CPH), poly[sebacic acids co-1,3-bis(p-carboxyphenoxy)propane] or P(SA:CPP), and poly(adipic anhydride).

Biodegradable materials include natural polymers and polymers derived therefrom, such as albumin, alginate, casein, chitin, chitosan, collagen, dextran, elastin, proteoglycans, gelatin and other hydrophilic proteins, glutin, zein and other prolamines and hydrophobic proteins, starch and other polysaccharides including cellulose and derivatives thereof (such as methyl cellulose, ethyl cellulose, hydroxypropyl cellulose, hydroxy-propyl methyl cellulose, hydroxybutyl methyl cellulose, carboxymethyl cellulose, cellulose acetate, cellulose propionate, cellulose acetate butyrate, cellulose acetate phthalate, cellulose acetate succinate, hydroxypropylmethylcellulose phthalate, cellulose triacetate, cellulose sulphate), poly-1-lysine, polyethylenimine, poly(allyl amine), polyhyaluronic acids, alginic acid, chitin, chitosan, chondroitin, dextrin or dextran), and proteins (such as albumin, casein, collagen, gelatin, fibrin, fibrinogen, hemoglobin).

In certain embodiments, a preferred biodegradable polymer includes a polyether, a polyester, a poly(ortho)ester, a polyketal, a polyamino acid, and a hydrogel. Various combinations, such as blends, of these can be used if desired.

In certain embodiments, preferred biodegradable polymers include hyaluronic acid and derivatives thereof, dextran and derivatives thereof, chitin, chitosan, albumin. Various combinations, such as blends, of these can be used if desired (e.g., blends of chitin, chitosan, and albumin in a wide variety of ratios).

Non-degradable (i.e., biostable) polymers include polyolefins such as polyethylene, polypropylene, polyurethanes, fluorinated polyolefins such as polytetrafluorethylene, chlorinated polyolefins such as poly(vinyl chloride), polyamides, acrylate polymers such as poly(methyl methacrylate), acrylamides such as poly(N-isopropylacrylamide), vinyl polymers such as poly(N-vinylpyrrolidone), poly(vinyl alcohol), poly(vinyl acetate), and poly(ethylene-co-vinylacetate), polyacetals, polycarbonates, polyethers such as based on poly(oxyethylene) and poly(oxypropylene) units, aromatic polyesters such as poly(ethylene terephthalate) and poly(propylene terephthalate), poly(ether ether ketone)s, polysulfones, silicone rubbers, epoxies, and poly(ester imide)s.

Preferred biodegradable polymers include polymers of lactide, caprolactone, glycolide, trimethylene carbonate, p-dioxanone, gamma-butyrolactone, or combinations thereof in the form of random or block copolymers. Preferred non-biodegradable polymers include polyesters, polyamides, polyurethanes, polyethers, vinyl polymers, and combinations thereof.

In certain embodiments, other polymers for use with the therapeutic agent, e.g., as a cap-coat or mixed therewith, include the following: a polymer with phosphoryl choline functionality to encourage ionic interactions, including but not limited to a methacrylate copolymer with a comonomer of Formula I; a polymer with multiple hydroxyl groups encouraging hydrogen bonding interaction with the therapeutic agents, including but not limited to that shown in Formula II; a polymer with acidic or basic groups encouraging acid-base interaction with the therapeutic agents, including but not limited to those shown in Formulas III and IV.

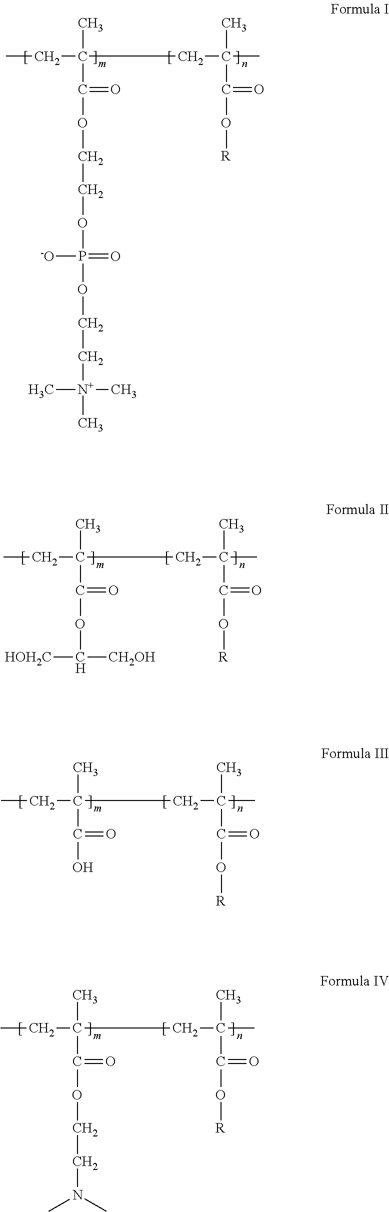

In the above formulas (I through IV), the R groups are independently C1 to C20 straight chain alkyl, C3 to C8 cycloalkyl, C2 to C20 alkenyl, C2 to C20 alkynyl, C2 to C14 heteroatom substituted alkyl, C2 to C14 heteroatom substituted cycloalkyl, C4 to C10 substituted aryl, or C4 to C10 substituted heteroatom substituted heteroaryl. In certain embodiments, m and n are individually integers from 1 to 20,000. In certain embodiments, m is an integer ranging from 10 to 20,000; from 50 to 15,000; from 100 to 10,000; from 200 to 5,000; from 500 to 4,000; from 700 to 3,000; or from 1000 to 2000. In certain embodiments, m is an integer ranging from 10 to 20,000; from 50 to 15,000; from 100 to 10,000; from 200 to 5,000; from 500 to 4,000; from 700 to 3,000; or from 1000 to 2000.

Other polymers for use with the therapeutic agent, e.g., as a cap-coat or mixed therewith, are shown below in Formulas V and VI:

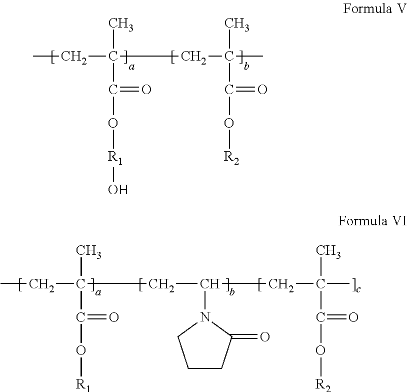

In the above formulas V, the R1 groups are independently C1 to C20 straight chain alkylene, C3 to C8 cycloalkylene, C2 to C20 alkenylene, C2 to C20 alkynylene, C2 to C14 heteroatom substituted alkylene, C2 to C14 heteroatom substituted cycloalkylene, C4 to C10 substituted arylene, or C4 to C10 substituted heteroatom substituted heteroarylene. In the above formulas V, the R2 groups are independently C1 to C20 straight chain alkyl, C3 to C8 cycloalkyl, C2 to C20 alkenyl, C2 to C20 alkynyl, C2 to C14 heteroatom substituted alkyl, C2 to C14 heteroatom substituted cycloalkyl, C4 to C10 substituted aryl, or C4 to C10 substituted heteroatom substituted heteroaryl. In certain embodiments, a is an integer ranging from 10 to 20,000; from 50 to 15,000; from 100 to 10,000; from 200 to 5,000; from 500 to 4,000; from 700 to 3,000; or from 1000 to 2000. In certain embodiments, b is an integer ranging from 10 to 20,000; from 50 to 15,000; from 100 to 10,000; from 200 to 5,000; from 500 to 4,000; from 700 to 3,000; or from 1000 to 2000.

In the above formula VI, the R1 and R2 groups are independently C1 to C20 straight chain alkyl, C3 to C8 cycloalkyl, C2 to C20 alkenyl, C2 to C20 alkynyl, C2 to C14 heteroatom substituted alkyl, C2 to C14 heteroatom substituted cycloalkyl, C4 to C10 substituted aryl, or C4 to C10 substituted heteroatom substituted heteroaryl. In certain embodiments, a is an integer ranging from 10 to 20,000; from 50 to 15,000; from 100 to 10,000; from 200 to 5,000; from 500 to 4,000; from 700 to 3,000; or from 1000 to 2000. In certain embodiments, b is an integer ranging from 10 to 20,000; from 50 to 15,000; from 100 to 10,000; from 200 to 5,000; from 500 to 4,000; from 700 to 3,000; or from 1000 to 2000. In certain embodiments, c is an integer ranging from 10 to 20,000; from 50 to 15,000; from 100 to 10,000; from 200 to 5,000; from 500 to 4,000; from 700 to 3,000; or from 1000 to 2000.

There are many polymer systems that can be used in delivering the one or more therapeutic agents described herein. Suitable examples are described, for example, in U.S. Pat. Pub. Nos. 2006/0275340 and 2005/0084515. Other examples of polymer systems include phosphorylcholine materials as described in U.S. Pat. No. 5,648,442 (Bowers et al.). U.S. Pat. Pub. Nos. 2006/0275340 and 2005/0084515 describe miscible polymer blends, wherein swellabilities of the miscible polymer blends are used as a factor in determining the combinations of polymers for a particular therapeutic agent.

U.S. Pat. Pub. Nos. 2002/0037358 and 2008/0021385 describe a therapeutic agent incorporated into a polymer coating of at least a portion of the balloon, from which the therapeutic agent is released as the polymer is slowly dissolved by the aqueous bodily fluids. U.S. Pat. Pub. Nos. 2009/0226502, 2009/0227948, and 2009/0227949 disclose a solvent-swellable polymer incorporating a therapeutic agent that forms a balloon wall or a coating disposed over the balloon. U.S. Pat. Pub. No. 2007/0298069 discloses a medical device comprising a polymeric carrier region that comprises a polymer, a therapeutic agent and a solubilising agent (solvent), wherein the polymeric carrier region can be a coating layer, but also a device component, or the entire device.

The polymer(s) used may be obtained from various chemical companies known to those with skill in the art. However, because of the presence of unreacted monomers, low molecular weight oligomers, catalysts, and other impurities, it may be desirable (and, depending upon the materials used, may be necessary) to increase the purity of the polymer used. The purification process yields polymers of better-known, purer composition, and therefore increases both the predictability and performance of the mechanical characteristics of the coatings. The purification process will depend on the polymer or polymers chosen. Generally, in the purification process, the polymer is dissolved in a suitable solvent. Suitable solvents include (but are not limited to) methylene chloride, ethyl acetate, chloroform, and tetrahydrofuran. The polymer solution usually is then mixed with a second material that is miscible with the solvent, but in which the polymer is not soluble, so that the polymer (but not appreciable quantities of impurities or unreacted monomer) precipitates out of solution. For example, a methylene chloride solution of the polymer may be mixed with heptane, causing the polymer to fall out of solution. The solvent mixture then is removed from the copolymer precipitate using conventional techniques.

The therapeutic agents may be linked by occlusion in the matrices of the polymer coating, bound by covalent linkages to the coating, or encapsulated in microcapsules (e.g., as described in U.S. Pat. No. 5,893,840) that are disposed within the one or more indentations of a balloon, and are themselves biodegradable.

The one or more therapeutic agents of the present disclosure also may be prepared in a variety of “paste” or gel forms that can be applied to the one or more indentations of a balloon, and optionally the entire surface of a balloon, as described herein. For example, within one embodiment of the disclosure, therapeutic coatings are provided which are liquid at one temperature (e.g., temperature greater than 37° C., such as 40° C., 45° C., 50° C., 55° C. or 60° C.), and solid or semi-solid at another temperature (e.g., ambient body temperature, or any temperature lower than 37° C.). Such “thermopastes” may be made utilizing a variety of techniques. Other pastes may be applied as a liquid, which solidify in vivo due to dissolution of a water-soluble component of the paste.

If it is desired to dispose one or more therapeutic agents in the indentations and the surface of the balloon between the indentations, various coating techniques, such as spray or dip coating, can be used. If it is desired to dispose one or more therapeutic agents only in the indentations, ink-jet printing or other pattern coating technique, can be used. Preferably, balloons of the present disclosure have one or more therapeutic agents disposed only in the indentations for greater control of the amount of therapeutic agent dispensed.

In accordance with this disclosure, tubing can be formed from desired balloon material using a conventional polymer extrusion process. Extruded tubing can then be blow-molded into a textured balloon using a mold, with various process variables of pressure and temperature. The mold may be made from a variety of materials, such as one or more metals or rigid polymers. The mold will have protrusions (peaks) on its inner surface, which will correspond to the desired texture pattern (valleys) on the balloon surface. This can be accomplished, for example, by placing a stent inside a mold.

In a preferred embodiment, for example a mold receives a tubular parison made of a polymeric material. The ends of the parison extend outwardly from the mold and one of the ends is sealed while the other end is affixed to a source of inflation fluid, typically nitrogen gas, under pressure. Clamps or “grippers” are attached to both ends of the parison so that the parison can be drawn apart axially in order to axially stretch the parison while at the same time said parison is capable of being expanded radially or “blown” with the inflation fluid. The radial expansion and axial stretch step or steps may be conducted simultaneously, or depending upon the polymeric material of which the parison is made, following whatever sequence is required to form a balloon. Failure to axially stretch the parison during the balloon forming process will result in a balloon that will have an uneven wall thickness and will exhibit a wall tensile strength lower than the tensile strength obtained when the parison is both radially expanded and axially stretched.

The polymeric parisons used in this disclosure are preferably drawn axially and expanded radially simultaneously within the mold. To improve the overall properties of the balloons formed, it is desirable that the parison is axially stretched and blown at temperatures above the glass transition temperature (Tg) of the polymeric material used. This expansion usually takes place at a temperature of 80° C. to 150° C., depending upon the polymeric material used in the process.

In accordance with this disclosure, based upon the polymeric material used, the parison is dimensioned with respect to the intended final configuration of the balloon. It is particularly important that the parison have relatively thin walls. The wall thickness is considered relative to the inside diameter of the parison which has wall thickness-to-inside diameter ratios of less than 0.6, and preferably between 0.57 and 0.09 or even lower. The use of a parison with such thin walls enables the parison to be stretched radially to a greater and more uniform degree because there is less stress gradient through the wall from the surface of the inside diameter to the surface of the outside diameter. By utilizing a parison which has thin walls, there is less difference in the degree to which the inner and outer surfaces of the tubular parison are stretched.

Preferably, the parison is drawn from a starting length L1 to a drawn length L2, which preferably is between about 1.10 to about 6 times the initial length L1. The tubular parison, which has an initial internal diameter ID1 and an outer diameter OD1, is expanded by the inflation fluid emitted under pressure to the parison to an internal diameter ID2, which is preferably 6 to 8 times the initial internal diameter ID1, and an outer diameter OD2, which is about equal to or preferably greater than about 3 times the initial outer diameter OD1. The parison is preferably subjected to between 1 and 5 cycles during which the parison is axially stretched and radially expanded with an elevated inflation pressure (i.e., a pressure sufficient to inflate the balloon), preferably an elevated pressure of at least 100 psi, and more preferably up to 500 psi. Nitrogen gas is the preferable inflation fluid for the radial expansion step.

Following the initial expansion step, the expanded parison is subjected to a “Heat Set” step, preferably while maintaining the elevated inflation pressure of at least 100 psi and more preferably up to 500 psi. The temperature chosen for the “Heat Set” step is one that induces crystallization and “freezes” or “locks” the orientation of the polymer chains which resulted from axially stretching and radially expanding the parison. The temperatures which can be used in this heat set step are therefore dependent upon the particular polymeric material used to form the parison and the ultimate properties desired in the balloon product (e.g., distensibility, strength, and compliancy). The temperatures chosen for this “Heat Set” step will more usually be above the temperature used during the initial expansion step but will be below the melting temperature of the melt temperature of the polymeric material from which the parison is formed. The heat set step ensures that the expanded parison and the resulting balloon will have temperature and dimensional stability.

The balloon thus formed may be removed from the mold, and affixed to a catheter. Following balloon formation, and prior to mounting on the catheter, one taper/cone region of the balloon is trimmed completely off the balloon (distal balloon region) while the other taper/cone region remains to form one of the bond regions. The other bond region of the balloon is part of the balloon body.

Preferably, one or more therapeutic agents will be precisely loaded into the valleys on the balloon surface by using ink-jet printing technology or by using a dispensing nozzle connected to the therapeutic agent reservoir. A balloon loaded with one or more therapeutic agents will be dried, pleated, folded, and wrapped as desired, before or after attaching it to the delivery catheter.

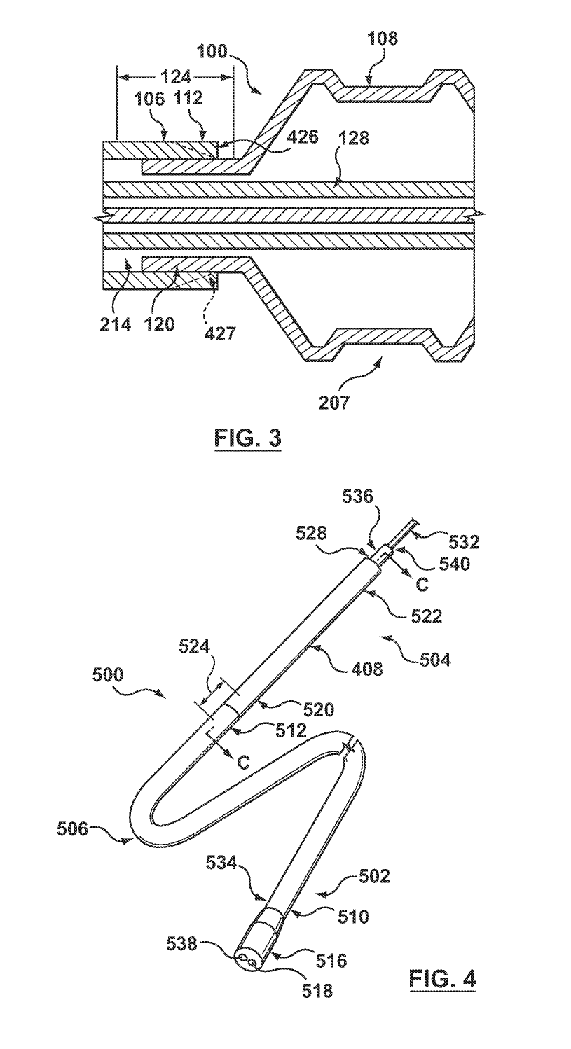

Referring now to FIGS. 2-3, an embodiment of a balloon catheter 100 according to the present disclosure is shown in an un-inflated state showing the indentations 207. Balloon catheter 100 includes a proximal portion 102, a distal portion 104, and a balloon 108 located at distal portion 104. Catheter 100 may be used for angioplasty procedures involving localized delivery of one or more therapeutic agents.

Catheter 100 includes an outer catheter shaft 106 which includes at least one continuous lumen 214 extending from at or near its proximal end 110 to at or near its distal end 112 in order to provide for balloon inflation. Balloon 108 is located at or near distal end 112 of shaft 106, and a hub 116 is located at or near proximal end 110 of shaft 106. Hub 116 includes a balloon inflation port 118 to allow fluid communication between inflation lumen 214 and balloon 108 so that the balloon 108 may be inflated. Hub 116 will serve in a conventional manner to provide a luer or other fitting in order to connect the catheter 100 to a source of balloon inflation, such as a conventional angioplasty activation device.

Balloon 108 includes a proximal end 120 and a distal neck end 122 and indentations 207. At joint transition area 124, proximal end 120 of balloon 108 is placed inside and joined to the distal end 112 of outer catheter shaft 106, as shown in FIG. 3. Balloon 108 may be joined to outer catheter shaft 106 in any conventional manner, such as laser welding, adhesives, heat fusing, ultrasonic welding, or any other mechanical method. The profile of balloon catheter 100 is reduced by placing the proximal end 120 of balloon 108 inside outer catheter shaft 106 because such a configuration allows for a smaller outer diameter at joint transition area 124.

FIG. 3 is an enlarged sectional view at the location along line B-B of FIG. 2, and illustrates joint transition area 124 of catheter 100. As previously mentioned, typically an angioplasty balloon is welded or otherwise mechanically attached to the outer catheter shaft by placing the proximal balloon neck on the outside of the catheter shaft. By placing the proximal balloon neck on the outside of the catheter shaft, the catheter presumably possesses a smoother profile for tracking the balloon to the treatment site since the “edge” created by the balloon to shaft joint is not pushed against the vessel wall while the balloon is being tracked through the patient's tortuous anatomy. However, it is found that the edge 426 created by proximal end 120 of balloon 108 being placed inside the outer catheter shaft 106 will not hinder the crossability and trackability of catheter 100 while balloon 108 is being tracked through the patient's tortuous anatomy. Rather, having the proximal end 120 of balloon 108 placed inside the outer catheter shaft allows for a smaller outer diameter at joint transition area 124 and thus provides a reduced catheter profile with improved crossability, trackability and stiffness.

In addition, edge 426 may be modified in order to create a tapered edge 427. Tapered edge 427 is illustrated as a dotted line in FIG. 3. Tapered edge 427 creates a smoother joint transition area 124 to ensure that the distal edge of the catheter shaft is not pushed against the vessel wall while being tracked through the patient's tortuous anatomy. Edge 426 may also be rounded or otherwise modified such as by a necking or thinning operation to create a smoother joint transition area 124.

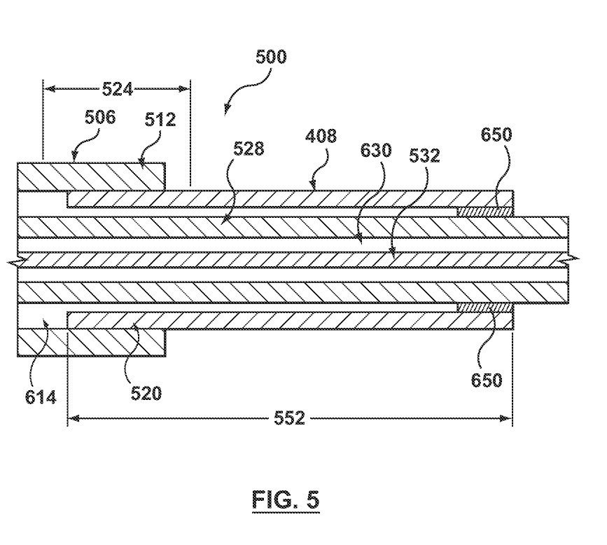

Now referring to FIGS. 4-5, another aspect of the present disclosure relates to a catheter 500 including a balloon 408 bonded to an outer catheter shaft 506, wherein the balloon is shown in a deflated state. FIG. 4 illustrates balloon catheter 500 having a proximal portion 502 and a distal portion 504 with inflatable balloon 408 located at distal portion 504. As best shown in FIG. 5, balloon 408 has a length 552.

Catheter 500 includes outer catheter shaft 506 which includes at least one continuous lumen 614 extending from at or near its proximal end 510 to at or near its distal end 512 in order to provide for balloon inflation. Balloon 408 is located at or near distal end 512 of shaft 506, and a hub 516 is located at or near proximal end 510 of shaft 506. Hub 516 includes a balloon inflation port 518 to allow fluid communication between inflation lumen 614 and balloon 408 so that the balloon 408 may be inflated. Hub 516 will serve in a conventional manner to provide a luer or other fitting in order to connect the catheter 500 to a source of balloon inflation, such as conventional angioplasty activation device.

FIG. 5 is an enlarged sectional view at the location along line C-C of FIG. 4, and illustrates joint transition area 524 of catheter 500. Balloon 408 includes a proximal end 520 and a distal end 522. At joint transition area 524, proximal end 520 of balloon 408 is placed inside and joined to the distal end 512 of outer catheter shaft 506. Balloon 408 may be joined to outer catheter shaft 506 in any conventional manner, such as laser welding, adhesives, heat fusing, ultrasonic welding, or any other mechanical method. The profile of balloon catheter 500 is reduced by placing the proximal end 520 of balloon 408 inside outer catheter shaft 506 because such a configuration allows for a smaller outer diameter at joint transition area 524. Transition area 524 in FIG. 5 may also be rounded or otherwise modified such as by a necking or thinning operation to create a smoother transition joint.

Catheter 500 includes an inner or guidewire shaft 528 disposed coaxially within outer catheter shaft 506. Inner shaft 528 includes at least one continuous lumen 630 extending from at or near its proximal end 534 to at or near its distal end 536 in order to provide a guidewire lumen 532. As illustrated in FIG. 4, inner shaft 528 may extend the entire length of catheter 500, with a proximal guidewire port 538 provided in hub 516 and a distal guidewire port 540 provided at the distal portion of catheter 500. The distal end 522 of balloon 408 is joined to the inner shaft 528 at joint 650 (FIG. 5). Balloon 508 may be joined to inner shaft 528 in any conventional manner, such as laser welding, adhesives, heat fusing, ultrasonic welding, or any other mechanical method.

The embodiments illustrated in FIGS. 2-5 include inner shaft (128 or 528) disposed within outer catheter shaft (106 or 506), with inner shaft (128 or 528) extending the entire length of catheter (100 or 500). Such a configuration is typically referred to as an over-the-wire (OTW) catheter. An OTW catheter's guidewire shaft runs the entire length of the catheter and is attached to, or enveloped within, an inflation shaft. Thus, the entire length of an OTW catheter is tracked over a guidewire during a PTCA procedure.

One skilled in the art can appreciate how the balloon to catheter joint of the present disclosure, described in detail above, may also be incorporated in a rapid exchange (RX) catheter. A RX catheter has a guidewire shaft that extends within only the distal-most portion of the catheter. Thus, during a PTCA procedure only the distal-most portion of a RX catheter is tracked over a guidewire.

Outer catheter shaft (106 or 506) may be formed of any appropriate polymeric material. In addition, inner shaft (128 or 528) may be made of any appropriate polymeric material. Non-exhaustive examples of material for outer catheter shaft (106 or 506) and inner shaft (128 or 528) include polyethylene, PEBAX, nylon or combinations of any of these, either blended or co-extruded. Preferred materials for shafts (106 or 506 and 128 or 528) are polyethylene, nylon, PEBAX, or co-extrusions of any of these materials.

Optionally, shafts (106 or 506 and 128 or 528) or some portion thereof may be formed as a composite having a reinforcement material incorporated within a polymeric body in order to enhance strength, flexibility, and/or toughness. Suitable reinforcement layers include braiding, wire mesh layers, embedded axial wires, embedded helical or circumferential wires, and the like. For example, at least a proximal portion of outer catheter shaft 106 may in some instances be formed from a reinforced polymeric tube. As a further alternative, at least a proximal portion of outer catheter shaft (106 or 506) may in some instances be formed from a metal, highly elastic, or super elastic hypotube material.

In any of the embodiments shown herein, inner shaft (e.g., 528 in FIG. 4) and outer catheter shaft (e.g., 506 in FIG. 4) may be arranged in various dual lumen configurations. For example, inner shaft and outer catheter shaft may be arranged in a coaxial dual lumen configuration. In the coaxial dual lumen configuration, an inflation lumen is created by a space between the outer surface of inner shaft and the inner surface of outer catheter shaft. This inflation lumen is in fluid communication with an interior of balloon such that balloon may be inflated. Other embodiments of balloon catheter may have guidewire lumen and inflation lumen in other dual lumen arrangements, such as a circular guidewire lumen above a D-shaped inflation lumen or a circular guidewire lumen set above a crescent-shaped inflation lumen.

### Illustrative Embodiments

1) A textured dilatation balloon comprising:
a non-compliant or semi-compliant balloon body comprising a proximal end, a distal end, and at least one indentation in the balloon body in an un-inflated state;
wherein the balloon body comprises a continuous polymer tube with an external surface comprising at least one therapeutic agent disposed within the at least one indentation prior to use.
2) The textured dilatation balloon of embodiment 1 wherein the external surface of the continuous tube of the balloon body further comprises at least one organic polymer disposed thereon.
3) The textured dilatation balloon of embodiment 2 wherein the organic polymer is a hydrophilic organic polymer.
4) The textured dilatation balloon of embodiment 2 or embodiment 3 wherein the organic polymer is a biodegradable organic polymer.
5) The textured dilatation balloon of embodiment 4 wherein the biodegradable organic polymer is selected from the group consisting of a polyether, a polyester, a poly(ortho)ester, a polyketal, a polyamino acid, a hydrogel, and combinations thereof.
6) The textured dilatation balloon of embodiment 4 wherein the biodegradable organic polymer is selected from the group consisting of hyaluronic acid, a hyaluronic derivative, dextran, a dextran derivative, chitin, chitosan, albumin, and combinations thereof.
7) The textured dilatation balloon of any one of embodiments 2 through 6 wherein the at least one therapeutic agent is located within the at least one indentation and the at least one organic polymer is disposed over the at least one therapeutic agent.
8) The textured dilatation balloon of any one of embodiments 2 through 6 wherein the at least one therapeutic agent is mixed with the at least one organic polymer to form a mixture that is disposed within the at least one indentation.
9) The textured dilatation balloon of any one of embodiments 1 through 8 wherein the at least one therapeutic agent is mixed with at least one excipient to form a mixture that is disposed within the at least one indentation.
10) The textured dilatation balloon of embodiment 9 wherein the excipient is selected from the group consisting of a fatty acid ester of polyethylene glycol, a polyethylene glycol-polyester block copolymer, a fatty acid mono- or di-ester of glycerol, a fatty acid mono-, di-, or poly-ester of trimethylol ethane or trimethylol propane or pentaerythritol, a sugar, a water-soluble polyol, cyclodextrin, a clathrate, and combinations thereof.
11) The textured dilatation balloon of any one of embodiments 1 through 10 wherein the at least one therapeutic agent is disposed only in the at least one indentation.
12) The textured dilatation balloon of any one of embodiments 1 through 11 wherein the balloon body comprises a proximal end, a distal end, and a plurality of indentations in the balloon body in an un-inflated state.
13) The textured dilatation balloon of embodiment 12 wherein the plurality of indentations are distributed symmetrically over the external surface of the continuous tube of the balloon body.
14) The textured dilatation balloon of any one of embodiments 1 through 13 wherein the at least one indentation comprises an inverted pyramid, an inverted truncated pyramid, a dimple, a groove, and combinations thereof.
15) The textured dilatation balloon of any one of embodiments 1 through 11 wherein the balloon body comprises a proximal end, a distal end, and one continuous indentation in the balloon body in an un-inflated state.
16) The textured dilatation balloon of any one of embodiments 1 through 15 wherein the continuous polymer tube of the balloon body comprises a polyethylene terephthalate, a polybutylene terephthalate, a polyamide, a polyether block amide, a polyblend comprising a polyamide, a polyblend comprising a polyethylene terephthalate, a polyblend comprising a polybutylene terephthalate, a multi-layer construction comprising a polyamide layer, a multi-layer construction comprising a polyethylene terephthalate layer, or a multi-layer construction comprising a polybutylene terephthalate layer.
17) The textured dilatation balloon of any one of embodiments 1 through 16 wherein the therapeutic agent is selected from the group consisting of an antiangiogenesis agent, an antirestenotic agent, an anticoagulant, an antiendothelin agent, an antimitogenic factor, an antioxidant, an antiplatelet agent, an antibiotic, an anti-inflammatory agent, an antiproliferative agent, an mTor inhibitor, an antineoplastic agent, an antisense oligonucleotide, an antithrombogenic agent, a gene therapy agent, a calcium channel blocker, a clot dissolving enzyme, a growth factor, a growth factor inhibitor, a nitric oxide releasing agent, a vasodilator, a virus-mediated gene transfer agent, a compound that affects microtubule development, a cell cycle inhibitor, an inhibitors of smooth muscle proliferation, an endothelial cell growth factor, a reverse cholesterol transport agonist, a reverse cholesterol transport antagonist, and combinations thereof.
18) The textured dilatation balloon of embodiment 17 wherein the therapeutic agent is selected from the group consisting of abciximab, angiopeptin, colchicine, eptifibatide, heparin, hirudin, lovastatin, methotrexate, streptokinase, paclitaxel, rapamycin, everolimus, deforolimus, ticlopidine, tissue plasminogen activator, trapidil, urokinase, and growth factors VEGF, TGF-beta, IGF, PDGF, FGF, and combinations thereof.
19) A method of delivering at least one therapeutic agent to a target site in a patient, the method comprising:
   providing a balloon catheter comprising a textured non-compliant or semi-compliant dilatation balloon of any one of embodiments 1 through 18;
   inserting the balloon catheter comprising the textured dilatation balloon into the target site of the patient; and
   inflating the textured balloon at the target site under conditions effective to deliver at least a portion of the therapeutic agent to the target site.
20) A method of delivering at least one therapeutic agent to a target site in a patient, the method comprising:
   providing a textured non-compliant or semi-compliant dilatation balloon comprising:
      a balloon catheter comprising a balloon body having a proximal end, a distal end, and at least one indentation in the balloon body in an un-inflated state;
      wherein the balloon body comprises a continuous polymer tube with an external surface having at least one therapeutic agent disposed within the at least one indentation prior to use;
   inserting the balloon catheter comprising the textured dilatation balloon into the target site of the patient; and
   inflating the textured balloon at the target site under conditions effective to deliver at least a portion of the therapeutic agent to the target site.
21) A method of making a textured dilatation balloon, the method comprising: providing a tubular parison comprising a polymeric material;
   providing a mold having one or more protrusions on its inner surface corresponding to the desired texture of the balloon surface;
   expanding the tubular parison to form an expanded parison in the mold and form a balloon body comprising one or more indentations; and
   applying one or more therapeutic agents into the one or more indentations.
22) The method of embodiment 21 wherein the balloon is a non-compliant or semi-compliant balloon.
23) The method of embodiment 21 or embodiment 22 wherein:
   expanding the tubular parison to form an expanded parison comprises axially stretching and radially expanding the tubular parison at a temperature above the Tg of the polymeric material and at an elevated inflation pressure.

The complete disclosures of the patents, patent documents, and publications cited herein are incorporated by reference in their entirety as if each were individually incorporated. Various modifications and alterations to this disclosure will become apparent to those skilled in the art without departing from the scope and spirit of this disclosure. It should be understood that this disclosure is not intended to be unduly limited by the illustrative embodiments and examples set forth herein and that such examples and embodiments are presented by way of example only with the scope of the disclosure intended to be limited only by the claims set forth herein as follows.

What is claimed is:

1) A textured dilatation balloon comprising:
a non-compliant or semi-compliant balloon body comprising a proximal end, a distal end, and at least one indentation in the balloon body in an un-inflated state;
wherein the balloon body comprises a continuous polymer tube with an external surface comprising at least one therapeutic agent disposed within the at least one indentation prior to use.

2) The textured dilatation balloon of claim 1 wherein the external surface of the continuous tube of the balloon body further comprises at least one organic polymer disposed thereon.

3) The textured dilatation balloon of claim 2 wherein the organic polymer is a hydrophilic organic polymer.

4) The textured dilatation balloon of claim 2 wherein the organic polymer is a biodegradable organic polymer.

5) The textured dilatation balloon of claim 4 wherein the biodegradable organic polymer is selected from the group consisting of a polyether, a polyester, a poly(ortho)ester, a polyketal, a polyamino acid, a hydrogel, and combinations thereof.

6) The textured dilatation balloon of claim 4 wherein the biodegradable organic polymer is selected from the group consisting of hyaluronic acid, a hyaluronic derivative, dextran, a dextran derivative, chitin, chitosan, albumin, and combinations thereof.

7) The textured dilatation balloon of claim 2 wherein the at least one therapeutic agent is located within the at least one indentation and the at least one organic polymer is disposed over the at least one therapeutic agent.

8) The textured dilatation balloon of claim 2 wherein the at least one therapeutic agent is mixed with the at least one organic polymer to form a mixture that is disposed within the at least one indentation.

9) The textured dilatation balloon of claim 1 wherein the at least one therapeutic agent is mixed with at least one excipient to form a mixture that is disposed within the at least one indentation.

10) The textured dilatation balloon of claim 9 wherein the excipient is selected from the group consisting of a fatty acid ester of polyethylene glycol, a polyethylene glycol-polyester block copolymer, a fatty acid mono- or di-ester of glycerol, a fatty acid mono-, di-, or poly-ester of trimethylol ethane or trimethylol propane or pentaerythritol, a sugar, a water-soluble polyol, cyclodextrin, a clathrate, and combinations thereof.

11) The textured dilatation balloon of claim 1 wherein the at least one therapeutic agent is disposed only in the at least one indentation.

12) The textured dilatation balloon of claim 1 wherein the balloon body comprises a proximal end, a distal end, and a plurality of indentations in the balloon body in an un-inflated state.

13) The textured dilatation balloon of claim 12 wherein the plurality of indentations are distributed symmetrically over the external surface of the continuous tube of the balloon body.

14) The textured dilatation balloon of claim 1 wherein the at least one indentation comprises an inverted pyramid, an inverted truncated pyramid, a dimple, a groove, and combinations thereof.

15) The textured dilatation balloon of claim 1 wherein the balloon body comprises a proximal end, a distal end, and one continuous indentation in the balloon body in an un-inflated state.

16) The textured dilatation balloon of claim 1 wherein the continuous polymer tube of the balloon body comprises a polyethylene terephthalate, a polybutylene terephthalate, a polyamide, a polyether block amide, a polyblend comprising a polyamide, a polyblend comprising a polyethylene terephthalate, a polyblend comprising a polybutylene terephthalate, a multi-layer construction comprising a polyamide layer, a multi-layer construction comprising a polyethylene terephthalate layer, or a multi-layer construction comprising a polybutylene terephthalate layer.

17) The textured dilatation balloon of claim 1 wherein the therapeutic agent is selected from the group consisting of an antiangiogenesis agent, an antirestenotic agent, an anticoagulant, an antiendothelin agent, an antimitogenic factor, an antioxidant, an antiplatelet agent, an antibiotic, an anti-inflammatory agent, an antiproliferative agent, an mTor inhibitor, an antineoplastic agent, an antisense oligonucleotide, an antithrombogenic agent, a gene therapy agent, a calcium channel blocker, a clot dissolving enzyme, a growth factor, a growth factor inhibitor, a nitric oxide releasing agent, a vasodilator, a virus-mediated gene transfer agent, a compound that affects microtubule development, a cell cycle inhibitor, an inhibitors of smooth muscle proliferation, an endothelial cell growth factor, a reverse cholesterol transport agonist, a reverse cholesterol transport antagonist, and combinations thereof.

18) The textured dilatation balloon of claim 17 wherein the therapeutic agent is selected from the group consisting of abciximab, angiopeptin, colchicine, eptifibatide, heparin, hirudin, lovastatin, methotrexate, streptokinase, paclitaxel, rapamycin, everolimus, deforolimus, ticlopidine, tissue plasminogen activator, trapidil, urokinase, and growth factors VEGF, TGF-beta, IGF, PDGF, FGF, and combinations thereof.

19) A method of delivering at least one therapeutic agent to a target site in a patient, the method comprising:
providing a balloon catheter comprising a textured non-compliant or semi-compliant dilatation balloon of claim 1;
inserting the balloon catheter comprising the textured dilatation balloon into the target site of the patient; and
inflating the textured balloon at the target site under conditions effective to deliver at least a portion of the therapeutic agent to the target site.

20) A method of delivering at least one therapeutic agent to a target site in a patient, the method comprising:
providing a textured non-compliant or semi-compliant dilatation balloon comprising:
a balloon catheter comprising a balloon body having a proximal end, a distal end, and at least one indentation in the balloon body in an un-inflated state;
wherein the balloon body comprises a continuous polymer tube with an external surface having at least one therapeutic agent disposed within the at least one indentation prior to use;
inserting the balloon catheter comprising the textured dilatation balloon into the target site of the patient; and
inflating the textured balloon at the target site under conditions effective to deliver at least a portion of the therapeutic agent to the target site.

21) A method of making a textured dilatation balloon, the method comprising:
providing a tubular parison comprising a polymeric material;
providing a mold having one or more protrusions on its inner surface corresponding to the desired texture of the balloon surface;
expanding the tubular parison to form an expanded parison in the mold and form a balloon body comprising one or more indentations; and
applying one or more therapeutic agents into the one or more indentations.

22). The method of claim 21 wherein the balloon is a non-compliant or semi-compliant balloon.

23) The method of claim 21 wherein:
expanding the tubular parison to form an expanded parison comprises axially stretching and radially expanding the tubular parison at a temperature above the Tg of the polymeric material and at an elevated inflation pressure.
